SEQUENCE LISTING (4 sequences)

SEQ ID NO: 1 - DNA, 758 bp, Mus musculus
  source 1..758: mol_type = other DNA
actcactgcccagagtctcatgaggcaatgagagtctgaagcaagcgctagcccccccacaccctggccggcagccaggggcagggtgggccaggaaggcttctgtttgaaactttgctggatcaggttcaggatgagaataaatgaggctctcctggcctggaggaggcagtcttgggaaccatgcagttccagagggacgtcccctccctgtggctgtggcggtcactatcactgctgctgctactgggcctggggttctcccagacccccagctacagggatgctgtgctccgagctgtggatgacttcaaccagcagtccctagacaccaatctctaccgtctcctggacctggatcctgagccccaaggggacgaggatccagatactcccaagtctgtgaggttccgagtgaaggagactgtatgtggcaaggcagagcggcagctacctgagcaatgtgccttcaaggaacagggggtggtgaagcagtgtatgggggcagtcaccctgaacccggccgctgattcttttgacatcagctgtaacgagcctggtgcacagccctttcggttcaagaaaatttcccggctggctggacttctccgcaaaggtggggagaagattggtgaaaagcttaagaaaattggccagaaaattaagaatttttttcagaaacttgtacctcagccagagtagtaggcctgccttggcctgtttctggattcctaaaataataaacttggtaaaagaaa

SEQ ID NO: 2 - protein, 172 aa, Mus musculus
MQFQRDVPSLWLWRSLSLLLLLGLGFSQTPSYRDAVLRAVDDFNQQSLDTNLYRLLDLDPEPQGDEDPDTPKSVRFRVKETVCGKAERQLPEQCAFKEQGVVKQCMGAVTLNPAADSFDISCNEPGAQPFRFKKISRLAGLLRKGGEKIGEKLKKIGQKIKNFFQKLVPQPE

SEQ ID NO: 3 - DNA, 745 bp, Homo sapiens
  source 1..745: mol_type = other DNA
gtcctgtgaagcaatagccaggggctaaagcaaaccccagcccacaccctggcaggcagccagggatgggtggatcaggaaggctcctggttgggcttttgcatcaggctcaggctgggcataaaggaggctcctgtgggctagagggaggcagacatggggaccatgaagacccaaagggatggccactccctggggcggtggtcactggtgctcctgctgctgggcctggtgatgcctctggccatcattgcccaggtcctcagctacaaggaagctgtgcttcgtgctatagatggcatcaaccagcggtcctcggatgctaacctctaccgcctcctggacctggaccccaggcccacgatggatggggacccagacacgccaaagcctgtgagcttcacagtgaaggagacagtgtgccccaggacgacacagcagtcaccagaggattgtgacttcaagaaggacgggctggtgaagcggtgtatggggacagtgaccctcaaccaggccaggggctcctttgacatcagttgtgataaggataacaagagatttgccctgctgggtgatttcttccggaaatctaaagagaagattggcaaagagtttaaaagaattgtccagagaatcaaggattttttgcggaatcttgtacccaggacagagtcctagtgtgtgccctaccctggctcaggcttctgggctctgagaaataaactatgagagcaatttcctcagg

SEQ ID NO: 4 - protein, 170 aa, Homo sapiens
MKTQRDGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

(19) United States
(12) Patent Application Publication
(10) Pub. No.: US 2024/0335483 A1
(43) Pub. Date: Oct. 10, 2024

(54) NEW METHOD TO TREAT AUTOIMMUNE DISEASES

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); UNIVERSITÉ PARIS CITÉ, Paris (FR); JIANGNAN UNIVERSITY, Wuxi, Jiangsu (CN)

(72) Inventors: Julien DIANA, Paris (FR); Jia SUN, Wuxi, Jiangsu (CN)

(21) Appl. No.: 18/579,644

(22) PCT Filed: Aug. 5, 2022
(86) PCT No.: PCT/EP2022/072060

§ 371 Date: Jan. 16, 2024

(30) Foreign Application Priority Data
Aug. 6, 2021 (EP) 21306102.1

(51) Int. Cl. A61K 35/744; A61K 35/00; A61P 3/10; A61P 37/06; C07K 14/47

(52) U.S. Cl. CPC A61K 35/744; A61P 3/10; A61P 37/06; C07K 14/4723; A61K 2035/115

(57) ABSTRACT

The present invention relates to the treatment of autoimmune diseases. The inventors determined that the cathelicidin related antimicrobial peptide (CRAMP) expression was defective in the colon of newborn NOD mice and that this defect was responsible for early dysbiosis. Dysbiosis stimulated the colonic epithelium to produce type I IFNs that pathologically imprinted the local immune system during the pre-weaning period. This miseducation of the immune system promoted the pancreatic autoimmune response and the development of diabetes. Increasing colonic CRAMP expression in newborn NOD mice, by local CRAMP treatment or by CRAMP-expressing probiotic, restored colonic homeostasis, and halted the diabetogenic response preventing autoimmune diabetes. Thus, they identified whether a defective colonic expression in the CRAMP antimicrobial peptide promotes autoimmunity in the pancreas. The use of CRAMP-expressing probiotic can be very helpful to treat autoimmune diseases and particularly autoimmune type 1 diabetes or obesity. Thus, the present invention relates to a recombinant CRAMP-expressing food-grade bacterium and its use in the treatment of autoimmune diseases.

### FIELD OF THE INVENTION

The present invention relates to a recombinant CRAMP-expressing food-grade bacterium and its use in the treatment of autoimmune diseases.

### BACKGROUND OF THE INVENTION

The role of the gut microbiota in the education of the immune system is now well established (1). Alteration of the microbiota, that causes the failure of immune education is likely an important contributor to the development of immune-related diseases not only in the gut but also in distant organs (2). However, our knowledge about the precise mechanisms at play must be improved before considering the use of microbiota-based therapy against immune-related diseases. One unresolved fundamental question is how imbalance in the gut microbiota arises and triggers a misguided immune system.

Antimicrobial peptides (AMPs) are ancestor members of the innate immune system of almost all living organisms. AMPs are highly expressed at the epithelial surfaces for the defense against invading pathogens, which thanks to their direct microbicide capacity (3). Beyond this anti-infective function, AMPs secreted by intestinal epithelial cells (IECs) are crucial for the construction and the maintenance of a balanced commensal microbiota (4). Impairment of AMP secretion by Paneth cells triggers a break-down of the microbiota homeostasis with bacterial penetration of the mucosal surface associated with intestinal inflammation (5).

We and others have highlighted the immunomodulatory role of AMPs expressed in the target tissue in the pathogenesis of autoimmune diseases (6), demonstrating as example their ability to directly promote regulatory T cells (Tregs) in the pancreas and to prevent type 1 diabetes (T1D) development in the non-obese diabetic (NOD) mouse model (7, 8). However, whether dysregulation of AMPs in the gut may participate in the development of T1D remains to be determined.

### SUMMARY OF THE INVENTION

In this study, the inventors determined that the cathelicidin related antimicrobial peptide (CRAMP) expression was defective in the colon of newborn NOD mice and that this defect was responsible for early dysbiosis. Dysbiosis stimulated the colonic epithelium to produce type I IFNs that pathologically imprinted the local immune system during the pre-weaning period. This miseducation of the immune system promoted the pancreatic autoimmune response and the development of diabetes. Increasing colonic CRAMP expression in newborn NOD mice, by local CRAMP treatment or by CRAMP-expressing probiotic, restored colonic homeostasis, and halted the diabetogenic response preventing autoimmune diabetes. Thus, they identified whether a defective colonic expression in the CRAMP antimicrobial peptide promotes autoimmunity in the pancreas. The use of CRAMP-expressing probiotic can be very helpful to treat autoimmune diseases and particularly autoimmune type 1 diabetes or obesity.

Thus, the invention relates to a recombinant CRAMP-expressing food-grade bacterium and its use in the treatment of autoimmune diseases. Particularly, the invention is defined by its claims.

### DETAILED DESCRIPTION OF THE INVENTION

### CRAMP-Expressing Probiotic and Use Thereof

First, the inventors clearly show that administration of a CRAMP-expressing probiotic corrects the microbiota and prevents autoimmune diabetes. Thus, this kind of probiotic can be very useful to treat or prevent an autoimmune disease in a subject in need thereof.

Thus, a first aspect of the invention relates to a recombinant CRAMP-expressing food-grade bacterium.

In a particular embodiment, the recombinant CRAMP-expressing food-grade bacterium is a recombinant CRAMP-expressing probiotic bacterium.

In another embodiment, the CRAMP peptide is an active fraction of the CRAMP peptide.

In another embodiment, the CRAMP peptide is the mouse CRAMP peptide and has a nucleic acids sequence as set for in the SEQ ID NO: 1 and an amino acids sequence as set for in the SEQ ID NO: 2.

In another embodiment, the CRAMP peptide is the human CRAMP peptide and has a nucleic acids sequence as set for in the SEQ ID NO: 3 and an amino acids sequence as set for in the SEQ ID NO: 4.

In a particular embodiment, the recombinant CRAMP-expressing probiotic bacterium can be a L. lactis strain or a Lactobacillus casei strain, a L. lactis htrA strain or a Lactobacillus plantarum strain or a Bifidobacterium longum strain.

In a particular embodiment, the recombinant CRAMP-expressing probiotic bacterium is a L. lactis strain, a Lactobacillus casei strain, a L. lactis htrA strain, a Lactobacillus plantarum strain or a Bifidobacterium longum strain.

In a particular embodiment said recombinant CRAMP-expressing probiotic bacterium is a recombinant CRAMP-expressing Lactococcus Lactis (as use herein the CRAMP-L. Lactis) deposited in accordance with the Budapest Treaty, on Aug. 6, 2021 at the COLLECTION NATIONALE DE CULTURES DE MICROORGANISMES (CNCM) under the accession number CNCM I-5727.

In a particular embodiment said recombinant CRAMP-expressing probiotic bacterium is a recombinant CRAMP-expressing Lactococcus Lactis deposited in accordance with the Budapest Treaty, on Aug. 6, 2021 at the COLLECTION NATIONALE DE CULTURES DE MICROORGANISMES (CNCM) under the accession number CNCM I-5727.

As used herein, the term “CRAMP” for “cathelicidin related antimicrobial peptide” has its general meaning in the art and denotes a peptide which kills bacteria but also binds to lipopolysaccharide (LPS) to neutralize its activity. CRAMP is highly expressed in bone marrow and its expression is reported to be up-regulated by inflammatory and infectious stimuli. The CRAMP peptide has a mouse Entrez Gene ID number: 12796 and a Uniprot protein ID number: P51437 and a human Entrez Gene ID number: 820 and a Uniprot protein ID number: P49913.

Nucleic acids sequence (ADNc) of the mouse CRAMP peptide (SEQ ID NO: 1):
actcactgcccagagtctcatgaggcaatgagagtctgaagcaagcgctagcccccccacaccctggccggcagccagg
ggcagggtgggccaggaaggcttctgtttgaaactttgctggatcaggttcaggatgagaataaatgaggctctcctggcctggagga
ggcagtcttgggaaccatgcagttccagagggacgtcccctccctgtggctgtggcggtcactatcactgctgctgctactgggcctgg
ggttctcccagacccccagctacagggatgctgtgctccgagctgtggatgacttcaaccagcagtccctagacaccaatctctaccgt
ctcctggacctggatcctgagccccaaggggacgaggatccagatactcccaagtctgtgaggttccgagtgaaggagactgtatgtg
gcaaggcagagcggcagctacctgagcaatgtgccttcaaggaacagggggtggtgaagcagtgtatgggggcagtcaccctgaa
cccggccgctgattcttttgacatcagctgtaacgagcctggtgcacagccctttcggttcaagaaaatttcccggctggctggacttctc
cgcaaaggtggggagaagattggtgaaaagcttaagaaaattggccagaaaattaagaatttttttcagaaacttgtacctcagccagag
tagtaggcctgccttggcctgtttctggattcctaaaataataaacttggtaaaagaaa
Amino acids sequence of the mouse CRAMP peptide (SEQ ID NO: 2):
MQFQRDVPSL WLWRSLSLLL LLGLGFSQTP SYRDAVLRAV DDFNQQSLDT
NLYRLLDLDP EPQGDEDPDT PKSVRFRVKE TVCGKAERQL PEQCAFKEQG
VVKQCMGAVT LNPAADSFDI SCNEPGAQPF RFKKISRLAG LLRKGGEKIG
EKLKKIGQKI KNFFQKLVPQ PE
Nucleic acids sequence (ADNc) of the human cathelicidin peptide (SEQ ID NO: 3):
gtcctgtgaagcaatagccaggggctaaagcaaaccccagcccacaccctggcaggcagccagggatgggtggatcag
gaaggctcctggttgggcttttgcatcaggctcaggctgggcataaaggaggctcctgtgggctagagggaggcagacatggggacc
atgaagacccaaagggatggccactccctggggcggtggtcactggtgctcctgctgctgggcctggtgatgcctctggccatcattg
cccaggtcctcagctacaaggaagctgtgcttcgtgctatagatggcatcaaccagcggtcctcggatgctaacctctaccgcctcctg
gacctggaccccaggcccacgatggatggggacccagacacgccaaagcctgtgagcttcacagtgaaggagacagtgtgcccca
ggacgacacagcagtcaccagaggattgtgacttcaagaaggacgggctggtgaagcggtgtatggggacagtgaccctcaaccag
gccaggggctcctttgacatcagttgtgataaggataacaagagatttgccctgctgggtgatttcttccggaaatctaaagagaagattg
gcaaagagtttaaaagaattgtccagagaatcaaggattttttgcggaatcttgtacccaggacagagtcctagtgtgtgccctaccctgg
ctcaggcttctgggctctgagaaataaactatgagagcaatttcctcagg
Amino acids sequence of the human cathelicidin peptide (SEQ ID NO: 4):
MKTQRDGHSL GRWSLVLLLL GLVMPLAIIA QVLSYKEAVL RAIDGINQRS
SDANLYRLLD LDPRPTMDGD PDTPKPVSFT VKETVCPRTT QQSPEDCDFK
KDGLVKRCMG TVTLNQARGS FDISCDKDNK RFALLGDFFR KSKEKIGKEF
KRIVQRIKDF LRNLVPRTES

As used herein, the term “food-grade bacterium” denotes a bacterium that is widely used in fermented foods and possesses a perfect safety profile recognized by the GRAS (Generally Recognized As Safe) and QPS (Qualified Presumption of Safety) status in USA and European Community, respectively. Such bacterium can be safely in functional foods or food additives with allegations concerning maintain in good health and well-being or prevention of disease. In some embodiments, the food-grade bacterium is a lactic acid bacterium. Lactic acid bacteria are among the most important groups of microorganisms used in food fermentations (M. P. Doyle et al., The Prokaryotes, pp 241-256). Lactic acid bacteria include as example Carnobacterium, Enterococcus, Lactobacillus, Lactococcus, Leuconostoc, Oenococcus, Pediococcus, Streptococcus, Tetragenococcus, Vagococcus, and Weissella. In a particular embodiment, the lactic acid bacterium is selected in the group consisting of Carnobacterium, Enterococcus, Lactobacillus, Lactococcus, Leuconostoc, Oenococcus, Pediococcus, Streptococcus, Tetragenococcus, Vagococcus, and Weissella. In some embodiments, the food-grade bacterium is a Lactobacillus selected from the group consisting of Lactobacillus plantarum, Lactobacillus casei, Lactobacillus rhamnosus, Lactobacillus crispatus, Lactobacillus fermentum, Lactobacillus johnsonii, Lactobacillus reuteri, and Lactobacillus amylovorus. In some embodiments, the food-grade bacterium is a Lactococcus Lactis. In a more particular embodiment, the food-grade bacterium is selected in the group consisting of Bifzdobacterium, Lactobacillus or Lactococcus.

As used herein, the term “probiotic bacterium” denotes a bacterium which ingested live in adequate quantities can exert beneficial effects on the human health. They are now widely used as a food additive for their health-promoting effects. Most of the probiotic bacteria are Lactic Acid Bacterium (LAB) and among them strains of the genera Lactobacillus and Bifidobacterium are the most widely used probiotic bacteria.

In a particular embodiment, the recombinant CRAMVP-expressing food-grade bacterium according to the invention comprises a defective auxotrophic gene, whereby survival of said bacterium is strictly dependent upon the presence of specific compounds.

In another embodiment, the auxotrophic gene according to the invention is the thyA gene encoding the thymidylate synthase.

In another embodiment, the auxotrophic gene according to the invention is the alanine racemase (alr) gene.

Inactivation of the thyA gene of the probiotic bacterium according to the invention renders it auxotrophic to thymidine which is absent from the gastrointestinal tract (GIT). This recombinant thyA mutant will be able to deliver its peptide of interest but will not survive and thus persist in GIT limiting its dissemination and conferring the requested biological containment for recombinant bacteria. Similar results can be obtained with alr gene.

In another preferred embodiment, the gene encoding for CRAMP is inserted in the thyA gene of the food-grade bacterium.

Particularly, the recombinant gene is located in the chromosome into the thyA gene locus which is thus inactivated by gene disruption. As used herein, the term “gene disruption” denotes disruption by insertion of a DNA fragment, disruption by deletion of the gene, or a part thereof, as well as exchange of the gene or a part thereof by another DNA fragment, and the disruption is induced by recombinant DNA techniques, and not by spontaneous mutation. Particularly, disruption is the exchange of the gene, or a part thereof, by another functional gene. recombinant CRAMP-expressing food-grade bacterium, the defective recombinant thyA gene is a non-reverting mutant gene.

As used herein, the term “non-reverting mutant” denotes that the reversion frequency is lower than 10⁻⁸, preferably the reversion frequency is lower than 10⁻¹⁰, even more preferably, the reversion frequency is lower than 10⁻¹², even more preferably, the reversion frequency is lower than 10⁻¹⁴, most preferably, the reversion frequency is not detectable using the routine methods known to the person skilled in the art.

A second aspect of the invention relates to a recombinant CRAMP-expressing food-grade bacterium according to the invention for use in the treatment of an autoimmune disease in a subject in need thereof.

In a particular embodiment, the invention relates to a recombinant CRAMP-expressing food-grade bacterium according to the invention for use in the treatment of a disease with disturbance of the microbiota in a subject in need thereof.

In another embodiment, the invention relates to a recombinant CRAMP-expressing food-grade bacterium according to the invention for use to improve or restore the function of the microbiota in a subject in need thereof.

In a particular embodiment, the autoimmune disease is an intra- and extra-intestinal dysbiosis-related disease. In a particular embodiment, the intra- and extra-intestinal dysbiosis-related disease is the obesity.

As used herein, the term “intra- and extra-intestinal dysbiosis-related diseases” included diseases with disturbance of the microbiota.

As used herein, the term “autoimmune diseases” include but is not limited to type 1 diabetes, rheumatoid arthritis, multiple sclerosis and autoimmune liver disease.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “subject” denotes a mammal, such as a rodent, a feline, a canine, and a primate. Particularly, the subject according to the invention is a human. Particularly, the subject suffers from an autoimmune disease like type 1 diabetes. Particularly, the subject has a family history with people afflicted with an autoimmune disease like type 1 diabetes, rheumatoid arthritis, multiple sclerosis and autoimmune liver disease or obesity. Particularly, the subject is a newborn baby, a baby or an infant with a mother having an autoimmune disease like type 1 diabetes, rheumatoid arthritis, multiple sclerosis and autoimmune liver disease or obesity. Particularly, the subject is a pregnant woman with an autoimmune disease like type 1 diabetes, rheumatoid arthritis, multiple sclerosis and autoimmune liver disease or obesity.

### Therapeutic Composition

Another object of the invention relates to a therapeutic composition comprising a recombinant CRAMP-expressing food-grade bacterium according to the invention.

In a particular embodiment, the invention relates to a therapeutic composition comprising a recombinant CRAMP-expressing food-grade bacterium according to the invention for use in the treatment of an autoimmune disease in a subject in need thereof.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

In a particular embodiment, the recombinant CRAMP-expressing food-grade bacterium according to the invention or the pharmaceutical composition according to the invention can be administrated to the subject in need thereof orally.

Particularly, the recombinant CRAMP-expressing food-grade bacterium according to the invention or the pharmaceutical composition according to the invention can be administrated to the subject in need thereof orally and incorporated to milk.

For the case of pregnant woman, the recombinant CRAMP-expressing food-grade bacterium according to the invention or the pharmaceutical composition according to the invention can be administrated to the pregnant woman several days before the delivery and particularly 5, 4, 3, 2 or days before the delivery.

For the case of a newborn baby, the recombinant CRAMP-expressing food-grade bacterium according to the invention or the pharmaceutical composition according to the invention can be administrated to the newborn baby several days after the delivery and particularly 5, 4, 3, 2 or days after the delivery.

Pharmaceutical compositions of the present invention may comprise a further therapeutic active agent. The present invention also relates to a kit comprising an agonist, antagonist or inhibitor of the expression according to the invention and a further therapeutic active agent.

For example, anti-diabetes agents may be added to the pharmaceutical composition as described below. For example insulin can be use in the pharmaceutical composition of the invention.

The invention will be further illustrated by the following figures and examples. However, these examples and figures should not be interpreted in any way as limiting the scope of the present invention.

### FIGURES

FIG. 1: Colonic CRAMP shapes microbiota composition preventing autoimmune diabetes. (A-B) Incidence of diabetes was followed in NOD mice treated with colonic CRAMP or scramble (sc)CRAMP (A) or NOD mice cross-fostered to colonic CRAMP-treated NOD dams (B), n=12 mice per group.

FIG. 2: CRAMP-expressing probiotic restores gut homeostasis preventing autoimmune diabetes and obesity. (A-B) Pregnant NOD mice were treated by oral gavage of CRAMP-expressing or conventional Lactococcus Lactis one day before delivery, the progeny was analyzed at different ages. (A) Incidence of diabetes was followed in the progeny, n=12 mice per group. (B) Body weight was followed from NOD mice progeny fed with high fat diet (HFD) or control diet from 8 weeks of age, n=12 mice per group.

### EXAMPLE

### Material & Methods

### Mice and Treatments

Female NOD, BALB/c, C57BL/6J, C57BL/6J camp−/− in different ages were used, bred and housed in specific pathogen-free conditions. In some experiments, male NOD mice were used as indicated in the figure legend. Recombinant mouse CRAMP1-39 and scrambled (sc)CRAMP1-39 were produced under aseptic conditions and provided after endotoxin removal processing (Innovagen). Newborn NOD mice were treated by intra-colonic treatment between 10 and 21 days of age every 3 days with CRAMP or scCRAMP (10 μg in 10 μL/mouse/injection) or vehicle (PBS-1% H₂O). For blocking TLR experiments, newborn NOD mice were treated between 7 and 14 days of age every 3 days with TLR2 inhibitor CU CTP22 (25 μg in 10 μL/mouse/injection) or TLR4 inhibitor TAK242 (25 μg in 10 μL/mouse/injection) or with TLR5 inhibitor TH1020 (20 μg in 10 μL/mouse/injection) or with vehicle (PBS 1% DMSO). All inhibitors are from Tocris. For modulating IFNα experiment, newborn NOD mice were treated between 7 and 14 days of age every 3 days with anti-IFNα receptor mAb (200 μg in 10 μL/mouse/injection) (BioXcell) or equivalent amount of isotype control (mouse IgGI) or gardiquimod (50 μg in 10 μL/mouse/injection) or vehicle (PBS-1% H₂O). For CRAMP-expressing probiotic experiment, pregnant NOD mice were given CRAMP-expressing Lactococcus lactis or conventional L. lactis (1010 colony forming units (cfu) in 100 μL PBS/mice) by oral gavage one day before delivery. For cross-fostering experiments, NOD pups were adopted immediately at birth by recipient mice (C57BL/6, BALB/c, CRAMP-treated NOD mice or NOD mice), that delivered the same day and pups from the two litters were maintained in the same cage. All animal experimental protocols were approved by the ethic committee for animal experimentation (APAFIS #3535-2015092416202090).

### Bacterial Strains

The L. lactis NZ9000 and L. lactis food-grade expression vector pNZ8148 were obtained from the in-house Culture Collections of Food Microbiology (CCFM) at the State Key Laboratory of Food Science and Technology, Jiangnan University (Wuxi, Jiangsu, China). L. lactis was transformed with pNZ8148-usp-Cath plasmid by electroporation. This plasmid contains secretion signal peptide usp45 and the nine-residue propeptides LEISSTCDA immediately upstream to CRAMP. L. lactis transformation was confirmed by PCR and western blot analysis. Bacteria were cultured in GM-17 broth (Sigma-Aldrich) with 0.5% glucose and erythromycin (10 μg/mL, Sigma-Aldrich) at 30° C. without aeration overnight, and then diluted in a fresh broth in 1/25 ratio, and incubated until A600 reached 0.4-0.5. Bacteria were then harvested by centrifugation (4000 g, 3 min), washed twice with sterilized water and resuspend in 100 μL PBS.

### Spontaneous Diabetes Incidence

NOD mice were treated as described above with CRAMP or scCRAMP, anti-IFNαR mAb, gardiquimod or were born from CRAMP-expressing L. lactis treated NOD mice or were cross-fostered by CRAMP-treated NOD mice. Overt diabetes was defined as two positive urine glucose tests, confirmed by a glycemia >200 mg.dl-1. Glukotest kit was purchased from Roche. Glucose tests and measure of glycemia were performed in a blind fashion.

### Preparation of Pancreatic Islets

Pancreata were perfused with a solution of collagenase P in HBSS-1% HEPES (0.75 mg.ml-1, Roche), then dissected free from surrounding tissues. Pancreata were digested at 37° C. for 8 min. Digestion was stopped by adding HBSS-10% FCS-1% EDTA followed by extensive washes. For flow cytometry analysis, islets were isolated on a discontinuous Ficoll® PM400 gradient (Sigma-Aldrich) and then isolated islets were handpicked to exclude contaminations from intrapancreatic lymph nodes. Cells were released from the islets by incubation at 37° C. for 6 min in non-enzymatic cell dissociation solution (Sigma-Aldrich). For RT-qPCR, to avoid potential contamination by exocrine tissue, islets were purified by handpicking in 3 consecutive baths of HBSS-10% FCS supplemented with 1% DNAse 1.

### Preparation of Lamina Propria Immune Cells

Immune cells from the colon were isolated using the Lamina Propria Dissociation Kit from Miltenyi (#130-097-410), combined with gentleMACS™ Octo dissociator with heaters (#130-096-427) according to the manufacturer's instructions.

### Isolation and Culture of Intestinal Epithelial Cells

Colons from 1-week-old mice were rinsed in HBSS (w/o), cleared from feces by holding with forceps and flushing with HBSS (w/o). Colons were longitudinally and then laterally cut into 1 cm pieces. Colon pieces were incubated at 37° C. for 10 min into a 15 mL tube containing 5 mL warm PBS supplemented by 30 mM EDTA. After mild shaking and wash at 250 g for 5 min at 4° C. in 5 mL HBSS (w/o), cell suspension well filtered on 100 μM cell strainers. After wash epithelial cells were cultured in 100 μL DMEM+F12 in 48-well plate overnight at 37° C. Supernatant were recovered by centrifugation at 5000 g for 10 min at 4° C. and immediately used to measure CRAMP expression by ELISA (MyBiosource, #MBS705604) or IFNα expression by ELISA (PBL, #42115-1).

### Flow Cytometry

Single cell suspensions were prepared from various tissues, surface staining was performed after FcγRII/III blocking (anti-CD16/CD32) for 5 min at 4° C. and were surface stained for 30 min at 4° C. Staining buffer was PBS containing 2% FCS, 0.5% EDTA and 0.1% sodium azide. For detection of the subsets of conventional dendritic cells, cells suspension were surface stained with anti-CD45, -TCRβ, -CD19-CD11c, -F4/80, -CD103, -CD11b. For detection of the diabetogenic CD8+ T cells, cells suspension were first incubated with tetramer-IGRP206-214 or tetramer-TUM for 1 h at RT, washed and surface stained with anti-CD45, -TCRβ, -CD19, -CD4, -CD8α for 30 min at 4° C. For regulatory T cell detection, cells were surface stained with anti-CD45, -TCRβ, -CD19, -CD4, -CD8□ and anti-LAP for 30 min at 4° C., after stained for Foxp3 expression followed the instruction of the Foxp3 staining kit (eBioscience). For detection of innate lymphoid cell subsets, Lin markers are a mix of anti-CD19, -CD5, -CD3ε, -B220, -CD11b, and -CD11c. For staining of transcription factors RoRγt, GATA3, T-bet, cells were stained using the True-Nuclear™ Transcription Factor kit (Biolegend). In all experiments dead cells were excluded using Fixable Viability Dye (eBioscience). Stained cells were analyzed on a Becton Dickinson Fortessa flow cytometer. Data were analyzed with Flowjo™ v10 software.

### Immunofluorescence Staining for CRAMP Expression

Colon were fixed in paraformaldehyde and embedded in paraffin, 4 micrometers sections were cut and deparaffinize, rehydrate and antigen retrieval was performed using the universal HIER antigen retrieval reagent (Abcam, #ab208572). Slides were blocked 30 min at RT using commercial blocking buffer (Abcam, #ab64226), and stained with anti-CRAMP pAb or rabbit serum (isotype control), incubate overnight at 4° C. After washing, anti-rabbit-AlexaFluor555 mAb (Invitrogen) were applied, incubate in dark for 1 h at RT. Nuclei were stained with DAPI. Image acquisition was performed on SFR Necker Imaging Facility using a Leica SP8 confocal microscope.

### Western Blot Analysis of CRAMP Expression

After overnight culture of colon epithelial cells, cell-free supernatants were analyzed by western blot. Electrophoresis was performed using 10%-20% Tricine Gels (Novex) in Tricine SDS running buffer (Novex). After separation, proteins were transferred to polyvinylidene difluoride (PVDF) membranes using iBlot™ 2 system (Invitrogen). Nonspecific binding to the PVDF membrane were saturated by exposure to 3% fat-free milk 0.5% BSA in TBST for 2 h before the membranes were incubated overnight at 4° C. with the anti-CRAMP pAb, followed by anti-rabbit-HRP pAb for 1 h at RT. The western Bright™ Quantum detection system (Diagomics) was used to visualize the immunoreactive bands. Band intensity quantification was performed using the GelQuantNET software.

### Measurement of TLR Ligand Levels in Colon Extract

Colons from 2-week-old mice were recovered in 100 μL PBS, cut in some pieces and homogenized by vortexing. Cell-free supernatant was recovered after centrifugation at 10 000 g for 10 min. A 100-fold dilution was used to measure TLR2 and TLR4 ligand levels in the supernatants using HEK-blue™ TLR reporter cells (Innovagen, #hkb-mtlr2 and #hkb-mtlr4) according to the manufacturer protocol.

### RT-qPCR

Total RNA was isolated using the Nucleospin RNA XS kit (Macherey-Nagel) from a minimum of 100 handpicked islets per mouse or using Nucleospin RNA kit (Macherey-Nagel) for colon tissue. RNA was reverse transcribed to synthesized cDNA using the high capacity cDNA reverse transcription kit (ThermoFisher) and measurements were performed by qPCR using Power SYBR® Green (ThermoFisher) on a 7900HT Fast System (Applied Biosystems). Resulting levels of fluorescence were submitted to relative quantification by normalization against a housekeeping gene (GAPDH) and expressed as 2-(ΔCT) values.

### RNAseq Gene Expression Profiling

Colon tissue was recovered from 5 mice from 5 independent cages per group. Total RNA were isolated from colon using the RNeasy Kit (QIAGEN) including a DNAse treatment step. RNA quality was assessed using RNA Screen Tape 6000 Pico LabChips with the Tape Station (Agilent Technologies) and RNA concentration was measured by spectrophometry using the Xpose (Trinean). RNAseq libraries were prepared starting from 1 μg of total RNA using the TruSeq Stranded mRNA LT Sample Prep Kit (Illumina) as recommended by the manufacturer. Half of the oriented cDNA produced from the poly-A+ fraction was PCR amplified (11 cycles). The RNAseq libraries were sequenced on an Illumina HiSeq2500 (Paired-End sequencing 130×130 bases, High Throughput Mode). A mean of 23 million of paired-end reads was produced per library sample (between 21 to 25 million of passing filter reads). The generated data were analyzed using the Ingenuity Pathway Analysis software (Qiagen).

### 16S rDNA Gene Sequencing and Sequence Analysis

Stools were recovered from 5 mice from 5 independent cages per group, and were stored at −80° C. in DNA/RNA Shield™ (Zymo Research, #R1100-250) immediately after emission before shipment to Zymo Research (ZymoBIOMICS Services). For CRAMP treatment experiments, pups from different litters were randomly mixed and then separate before treatment with CRAMP or scCRAMP. The samples were processed and analyzed with the ZymoBIOMICS® Targeted Sequencing Service for Microbiome Analysis (Zymo Research, Irvine, CA). DNA Extraction: One of three different DNA extraction kits was used depending on the sample type and sample volume. In most cases, the ZymoBIOMICS® DNA Miniprep Kit (Zymo Research, Irvine, CA) was used. For low biomass samples, such as skin swabs, the ZymoBIOMICS® DNA Microprep Kit (Zymo Research, Irvine, CA) was used as it permits for a lower elution volume, resulting in more concentrated DNA samples. For a large sample volume, the ZymoBIOMICS@-96 MagBead DNA Kit (Zymo Research, Irvine, CA) was used to extract DNA using an automated platform. Targeted Library Preparation: Bacterial 16S ribosomal RNA gene targeted sequencing was performed using the Quick-16S™ NGS Library Prep Kit (Zymo Research, Irvine, CA). The bacterial 16S primers amplified the V1-V2 or V3-V4 region of the 16S rRNA gene. These primers have been custom-designed by Zymo Research to provide the best coverage of the 16S gene while maintaining high sensitivity. Fungal ITS gene targeted sequencing was performed using the Quick-16S™ NGS Library Prep Kit with custom ITS2 primers substituted for 16S primers. The sequencing library was prepared using an innovative library preparation process in which PCR reactions were performed in real-time PCR machines to control cycles and therefore prevent limit PCR chimera formation. The final PCR products were quantified with qPCR fluorescence readings and pooled together based on equal molarity. The final pooled library was cleaned up with the Select-a-Size DNA Clean & Concentrator™ (Zymo Research, Irvine, CA), then quantified with TapeStation®(Agilent Technologies, Santa Clara, CA) and Qubit® (Thermo Fisher Scientific, Waltham, WA). Sequencing: The final library was sequenced on Illumina® MiSeq™ with a v3 reagent kit (600 cycles). The sequencing was performed with >10% PhiX spike-in. Bioinformatics Analysis: Unique amplicon sequences were inferred from raw reads using the DADA2 pipeline. Chimeric sequences were also removed with the DADA2 pipeline. Taxonomy assignment was performed using Uclust from Qiime v.1.9.1 with the Zymo Research Database, a 16S database that is internally designed and curated, as reference. Composition visualization, alpha-diversity, and beta-diversity analyses were performed with Qiime v.1.9.1. If applicable, taxonomy that have significant abundance among different groups were identified by LEfSe using default settings. Other analyses such as heatmaps, Taxa2SV_deomposer, and PCoA plots were performed with internal scripts.

### Statistical Analysis

Diabetes incidence was plotted according to the Kaplan-Meier method. Incidences between each group were compared with the log-rank test. Reported values are median +/− interquartile range as indicated. Comparison between each group was performed using the non-parametric Mann-Whitney U-test or one-way ANOVA when more than 2 groups were compared. P values <0.05 were considered statistically significant. All data were analyzed using GraphPad Prism v6 software.

### Results

### Cathelicidin Expression is Defective in the Colon of Newborn NOD Mice

AMPs are highly expressed by the gut epithelium where they play a critical role in the construction of a balanced microbiota, which is essential for educate the immune system and promote intestinal homeostasis (9). We analyzed the transcriptome and the microbiota in the colon from 2-week-old autoimmune-prone NOD mice compared with the non-autoimmune-prone BALB/c and C57BL/6 strains. We focused our study on the colon as it represents the preferential site of production of immune-modulatory metabolites by the microbiota (10). By RNAseq analysis, we observed that NOD mice harbored a specific pattern of AMP expression compared with control strains with a defective expression of cathelicidin-related antimicrobial peptide (CRAMP) (data not shown), which also confirmed by RT-qPCR (data not shown). A similar observation was performed in the ileum (data not shown). Western blot experiments on colon explant culture confirmed that CRAMP was poorly expressed in 3-day-old NOD mice compared with control strains (data not shown). Interestingly, CRAMP expression was also lower in NOD female mice compared with male NOD mice (data not shown) suggesting that a protective role for CRAMP against autoimmune diabetes since male NOD mice are partially protected against the disease. Using confocal microscopy and flow cytometry, we confirmed that CRAMP was expressed by the IECs in the newborn's colon and that CRAMP expression was defective in female NOD mice (data not shown). CRAMP is the only AMP constitutively expressed in the prenatal intestine and is proposed to be critical for the construction of the gut microbiota (11). We analyzed the microbiota by 16S RNA sequencing from the colon content of 2-week-old mice (data not shown). We observed that the microbiota of the NOD mice differed from the microbiota of BALB/c and C57BL/6 mice, which had a lower abundance of bacteria associated with prevention of T1D such as the genera Akkermansia, Clostridium and S24_7 (12). These data prompted us to investigate whether colonic CRAMP influences the construction of the microbiota and the development of diabetes.

### Colonic Cathelicidin Shapes the Microbiota and Prevents Autoimmune Diabetes

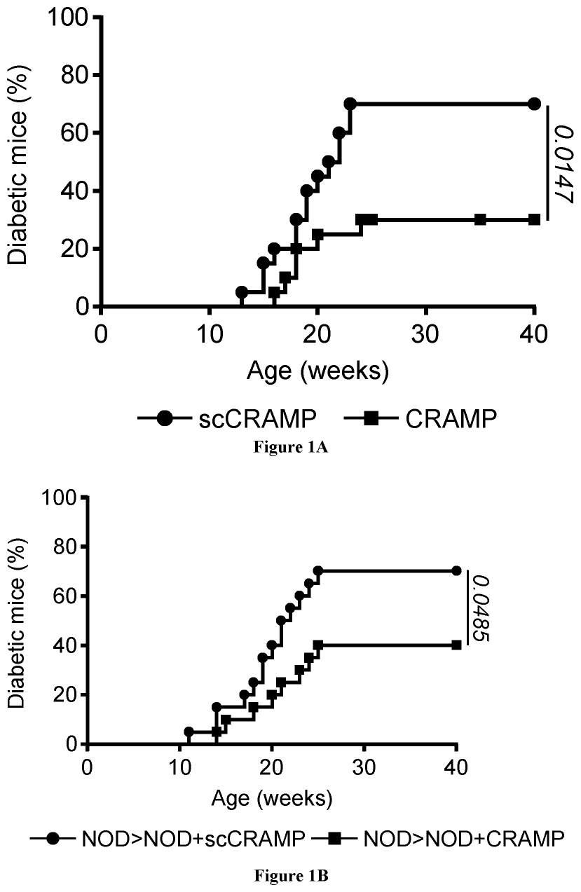

Ten-day-old female NOD mice were treated by intracolonic administration of CRAMP every 3 days until 21 days of age. Scramble (sc) CRAMP peptide was used as control. Microbiota composition was analyzed at 5 weeks of age when microbiota composition is stabilized after the weaning period (2). We observed that CRAMP treatment of the newborn NOD mice changed microbiota composition in the adult mice (data not shown). Remarkably, we observed an increased abundance of the genera Lactobacillus and Bifidobacterium, that were associated with T1D protection (13, 14) and a decreased abundance of the genus Alistipes that was associated with T1D development (12, 15). We also took advantage of the CRAMP-deficient C57BL/6 mice to confirm the impact of CRAMP expression on microbiota composition. We observed that the microbiota of 5-week-old CRAMP-deficient mice differed from the littermate controls with a decreased abundance of genera associated with T1D protection such as Lachnoclostridium, Akkermansia, Bacteroides or Bifidobacterium (12, 15, 16) and an increased abundance of the genera Alistipes and Roseburia, associated with T1D development (data not shown). Then, we analyzed whether intracolonic CRAMP administration in newborn NOD mice later impacted on the development of the autoimmune response in the pancreas in prediabetic 12-week-old NOD mice. CRAMP treatment in the newborn mice increased the frequency, but not the absolute number, of Treg cells in the pancreas and colon of adult NOD mice, but not in the pancreatic (P) or mesenteric (M) lymph nodes (LN), or the spleen (data not shown). Accordingly, the frequency and absolute number of pancreatic CD8+ effector T cells specific for the β-cell antigen islet-specific glucose-6-phosphatase catalytic subunit related protein (IGRP)206-214 was decreased in the pancreas and PLN in the adult mice after CRAMP treatment (data not shown). In line with these results, intracolonic CRAMP treatment in newborn NOD mice significantly prevented the development of diabetes (FIG. 1A). Finally, cross-fostering experiment showed that newborn NOD mice from untreated NOD dams fostered with NOD dams treated by CRAMP between 10 and 21 days of age results in a decreased incidence of diabetes in the adopted NOD mice (FIG. 1). Together, our data supported that a defective expression of CRAMP in the colon of newborn NOD mice is associated with early dysbiosis and restoring CRAMP expression in the newborn mice modified the microbiota in the adult inhibiting the development of diabetes.

### Newborn NOD Mice Exhibit Aberrant Type I IFN Signature in the Colon

To decipher the mechanism linking CRAMP-deficiency, dysbiosis and diabetes, we analyzed the transcriptome of the colon from 2-week-old NOD mice. RNAseq and RT-qPCR analyses revealed the presence of an aberrant type I IFN signature in the NOD mice compared with the control strains (data not shown). Type I IFN signature was transient since it was not observed in pre-diabetic 10-week-old NOD mice (data not shown). The presence of IFNα in the colon of 2-week-old NOD mice was confirmed by ELISA on colon explant cultures (data not shown) and was associated with an increased expression of Lcn2 mRNA, which is a classical marker of intestinal inflammation (17) (data not shown). Previous studies demonstrated that regular microbial colonization during the first postnatal weeks in non-autoimmune mouse strains accompanied with the down-regulation of type I IFN and TLR expression in the intestine, a mechanism required to prevent inflammatory response with respect to growing microbial exposure (18, 19). We performed cross-fostering experiments of newborn NOD mice with BALB/c or C57BL/6 dams and observed that colonic type I IFN signature and Lcn2 expression decreased in 2-week-old NOD mice adopted by control strain dams supporting that aberrant type I IFN expression was dependent on the NOD microbiota (data not shown). Additionally, cross-fostering of newborn NOD mice by BALB/c or C57BL/6 dams slightly reduced the incidence of diabetes (data not shown). Type I IFN signature was reduced by colonic CRAMP treatment of 10-day-old NOD mice, likely due to the ability of CRAMP to correct the microbiota (data not shown). This conclusion was also supported by the presence of a type I IFN signature in the colon of CRAMP-deficient C57BL/6 mice at 2 weeks of age (data not shown). Type I IFN expression by colonic epithelial cells can be stimulated by various microbiota products via specific TLRs20. RT-qPCR analysis revealed an overexpression of TLR2 and TLR4 but not TLR5 in the colon of 2-week-old NOD mice compared with non-autoimmune mouse strains (data not shown). Besides, using reporter cells, we observed that the levels of TLR2 and TLR4 ligands were higher in colon extract from 2-week-old NOD mice compared with non-autoimmune strains (data not shown) suggesting that the divergent NOD microbiota carries higher immunostimulatory capacity. Importantly, intracolonic CRAMP treatment reduced the levels of TLR2 and TLR4 ligands in the colon in line with the modifying effect of CRAMP on the microbiota composition (data not shown). Using specific antagonists, we finally demonstrated that colonic type I IFN signature in the newborn NOD mice was dependent on TLR2 and TLR4 but not TLR5 (data not shown). Altogether, our data support that dysbiotic microbiota in the colon of newborn NOD mice sustained, via TLR2 and TLR4, an aberrant type I IFN expression by the colonic epithelium that is prevented by CRAMP treatment through the modification of the microbiota.

### Aberrant Colonic Type I IFN Expression Miseducates the Immune System

Various immune deviations have been described in the intestine of adult pre-diabetic NOD mice. However, whether these immune abnormalities originated from earlier events take place during education of immune system remained to be determined. We observed that the colon from pre-diabetic 6-week-old NOD mice had a lower frequency of tolerogenic CD103+CD11b− cDC1s and a higher frequency of inflammatory CD103+CD11b+ cDC2s compared with non-autoimmune strains (data not shown). Importantly, blocking type I IFN expression in the newborn NOD mice by intracolonic anti-IFNαR mAb treatment (data not shown), decreased the frequency of cDC2s in the colon of adult NOD mice (data not shown). CDC2s are characterized by their ability to produce IL-23 (21), favoring IL-17-secreting type 3 ILCs (ILC3s) (22). RT-qPCR analysis confirmed a higher expression of IL-23 in NOD colon compared with non-autoimmune strains (data not shown). We observed that the frequency of ILC3s was higher in the colon of 6-week-old NOD mice compared with control strains while ILC2 frequency was lower (data not shown). Importantly, this imbalance in ILC subsets observed in the adult NOD mice originated from the aberrant type I IFN expression observed in the newborn's colon as shown by the use of anti-IFNαR blocking mAb that reduced the frequency of ILC3s (data not shown). At the cytokine level, adult NOD colon was characterized by a high expression of inflammatory cytokines IL-17, IL-6 and TNFα and a similar expression of regulatory cytokines TGFβ and IL-10 compared with control strains, but inhibition of type I IFN expression in the newborn NOD colon inhibited the expression of inflammatory cytokines in the adult (data not shown). According to its ability to suppress aberrant type I IFN expression, CRAMP treatment of newborn NOD mice prevented the overexpression of inflammatory cytokines in the adult NOD colon (data not shown). Importantly, CRAMP treatment of adult NOD mice was inefficient at changing the cytokine profile in the colon supporting its impact on immune education during the preweaning period (data not shown). Together, these data supported that dysbiosis-induced type I IFN expression in the newborn NOD mice pathologically imprints the colonic immune system towards an inflammatory profile.

### Aberrant Colonic Type I IFN Expression Supports Diabetes Development in NOD Mice

Intestinal inflammation has been associated with the development of autoimmune diabetes (14). We sought to determine whether type I IFN-dependent inflammation in the colon may affect the autoimmune response in the pancreas. We observed that inhibiting type I IFN expression in the colon of newborn NOD mice drastically decreased the mRNA expression of inflammatory cytokines in the pancreas of pre-diabetic 12-week-old NOD mice (data not shown). However, the expression of regulatory cytokines TGFβ or IL-10 were not increased. At the cellular level, inhibition of colonic type I IFNs increased the frequency but not the absolute number of Treg cells in the pancreas and colon but not in the PLN, MLN or spleen in the adult NOD mice (data not shown). In parallel, the frequency and absolute number of diabetogenic CD8+ effector T cells specific for IGRP206-214 was decreased in the pancreas and PLN of adult NOD mice (data not shown). To better support the diabetogenic role of colonic type I IFN expression in newborn NOD mice, we took advantage of the male NOD mice that present a less aggressive diabetogenic response. We performed intracolonic administration of gardiquimod in 10-day-old male NOD mice to induce the expression of type I IFNs (data not shown) and we analyzed the immune response in the pancreatic islets at 12 weeks of age. We observed that boosting the colonic expression of IFNα decreased the frequency of pancreatic LAP+ Treg cells in the adult NOD mice (data not shown). Accordingly, modulating type I IFN expression in newborn NOD mice either using blocking anti-IFNαR mAb treatment in female or gardiquimod treatment in male, reduced or increased diabetes incidence, respectively (data not shown). Finally, cross-fostering of newborn NOD mice by NOD dams previously treated with CRAMP at 10 days of age, prevented the aberrant type I IFN signature in the newborns (data not shown), which corresponded with the decrease of diabetes development (FIG. 1). Altogether, these data supported that aberrant type I IFN expression, which is responsible for a pathological imprinting of the immune system promotes the development of the diabetogenic response.

### CRAMP-Expressing Probiotic Restores Gut Homeostasis Preventing Autoimmune Diabetes

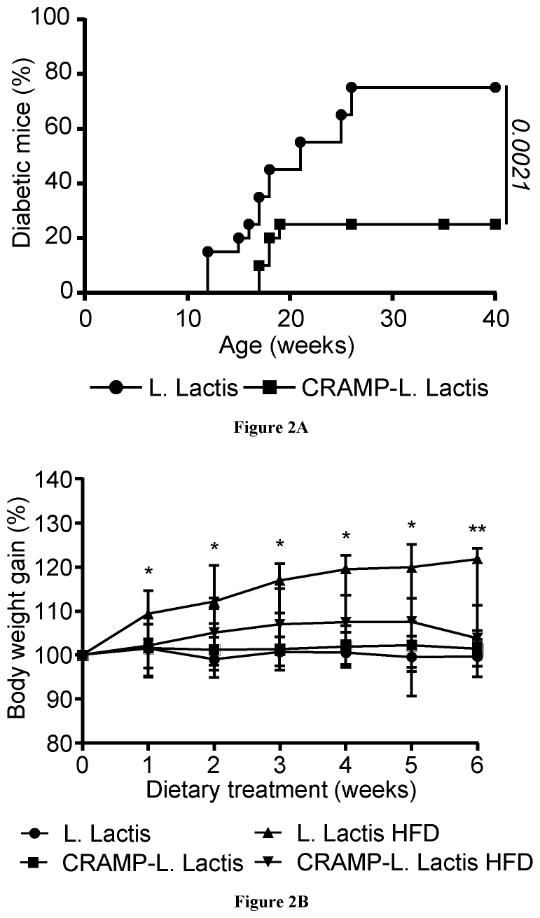

With the objective to develop a therapeutic approach potentially appropriate to prevent T1D in at-risk individuals, we generated a CRAMP-expressing Lactococcus Lactis that was administrated orally to NOD dams one day before delivery in order to naturally transfer the probiotic to the progeny at birth (data not shown). Conventional L. Lactis was used as control. The probiotic was detected in the progeny at birth until weaning (data not shown) and the probiotic efficiently produced CRAMP in the colon of 1-week-old NOD mice (data not shown). Importantly, the presence of CRAMP-expressing L. Lactis in the newborns modified the microbiota composition in adult NOD mice (data not shown). We observed an increased abundance of the “beneficial” order Bacteroidales, whereas several genera associated with T1D or obesity were decreased i.e. Roseburia, Acetatifactor, Lachnoclostridium, Anaerotruncus, Tyzzerella (23, 24). We also observed, while not statistically significant, an increased abundance of Akkermansia (data not shown), which is a bacteria associated with T1D protection and gut homeostasis in mice and human (25, 26). Mucus layer has been shown to be altered in the NOD mice (27), analyzing mucin expression in the colon at 6 weeks of age, we showed that CRAMP-expressing L. Lactis increased the expression of the immunoregulatory Muc1 and decreased the expression of the inflammatory Muc4 while Muc2 expression was unchanged compared with L. Lactis-treated NOD mice (data not shown). Immunologically, CRAMP-expressing L. Lactis decreased type I IFN signature in the colon of newborn NOD mice (data not shown) and decreased IL-17 expression while increased IL-10 expression in the colon of adult NOD mice (data not shown). At 12 weeks of age, CRAMP-expressing L. Lactis increased the frequency, but not the absolute number, of Treg cells in the pancreas and PLN but not in the spleen (data not shown) while the frequency and absolute number of diabetogenic IGRP206-214-specific CD8+ T cells was reduced in the pancreas (data not shown). Accordingly, CRAMP-expressing L. Lactis in the newborn NOD mice significantly prevented immune infiltration of the pancreatic islets and the development of diabetes in the adult NOD mice (FIG. 2A). Finally, extending the anti-diabetogenic effect of CRAMP-expressing L. Lactis, and according to the modifications observed on the microbiota, we observed that the probiotic reduced weight gain and the development of body fat mass in NOD mice under high-fat-diet-feeding condition (FIG. 2B). Altogether, our data supported that restoring CRAMP expression in the colon of NOD mice at birth is an efficient strategy to prevent the development of autoimmune diabetes.

### CONCLUSION

Altogether, our data support that in the pro-autoimmune NOD mice, cathelicidin-deficiency induces microbiota alterations, pathological imprinting of the immune system, hence contributing, with extra-intestinal factors, to the development of autoimmune diabetes. Besides, our study of CRAMP-deficient C57BL/6 mice suggests that a similar mechanism may be at play even in non-autoimmune prone genetic background and may contribute to the development of various intra- and extra-intestinal dysbiosis-related diseases. Consequently, the use of cathelicidin-expressing probiotic during pregnancy may be an efficient strategy to prevent the development of dysbiosis-related diseases in the offspring (28).

### REFERENCES

1. Gensollen T, Iyer S S, Kasper D L, et al. How colonization by microbiota in early life shapes the immune system. Science 2016; 352:539-44.
2. Al Nabhani Z, Eberl G. Imprinting of the immune system by the microbiota early in life. Mucosal Immunol 2020; 13:183-189.
3. Zasloff M. Antimicrobial peptides of multicellular organisms. Nature 2002; 415:389-95.
4. Salzman N H, Bevins C L. Dysbiosis—a consequence of Paneth cell dysfunction. Semin Immunol 2013; 25:334-41.
5. Ramanan D, Tang M S, Bowcutt R, et al. Bacterial sensor Nod2 prevents inflammation of the small intestine by restricting the expansion of the commensal Bacteroides vulgatus. Immunity 2014; 41:311-24.
6. Liang W, Diana J. The Dual Role of Antimicrobial Peptides in Autoimmunity. Front Immunol 2020; 11:2077.
7. Miani M, Le Naour J, Waeckel-Enee E, et al. Gut Microbiota-Stimulated Innate Lymphoid Cells Support beta-Defensin 14 Expression in Pancreatic Endocrine Cells, Preventing Autoimmune Diabetes. Cell Metab 2018; 28:557-572 e6.
8. Sun J, Furio L, Mecheri R, et al. Pancreatic beta-Cells Limit Autoimmune Diabetes via an Immunoregulatory Antimicrobial Peptide Expressed under the Influence of the Gut Microbiota. Immunity 2015; 43:304-17.
9. Bevins C L, Salzman N H. Paneth cells, antimicrobial peptides and maintenance of intestinal homeostasis. Nat Rev Microbiol 2011; 9:356-68.
10. Pereira F C, Berry D. Microbial nutrient niches in the gut. Environ Microbiol 2017; 19:1366-1378.
11. Renz H, Brandtzaeg P, Hornef M. The impact of perinatal immune development on mucosal homeostasis and chronic inflammation. Nat Rev Immunol 2011; 12:9-23.
12. Dedrick S, Sundaresh B, Huang Q, et al. The Role of Gut Microbiota and Environmental Factors in Type 1 Diabetes Pathogenesis. Front Endocrinol (Lausanne) 2020; 11:78.
13. Calcinaro F, Dionisi S, Marinaro M, et al. Oral probiotic administration induces interleukin-10 production and prevents spontaneous autoimmune diabetes in the non-obese diabetic mouse. Diabetologia 2005; 48:1565-75.
14. Pearson J A, Agriantonis A, Wong F S, et al. Modulation of the immune system by the gut microbiota in the development of type 1 diabetes. Hum Vaccin Immunother 2018:1-17.
15. Vatanen T, Franzosa E A, Schwager R, et al. The human gut microbiome in early-onset type 1 diabetes from the TEDDY study. Nature 2018; 562:589-594.
16. Forbes J D, Van Domselaar G, Bernstein C N. The Gut Microbiota in Immune-Mediated Inflammatory Diseases. Front Microbiol 2016; 7:1081.
17. Stallhofer J, Friedrich M, Konrad-Zerna A, et al. Lipocalin-2 Is a Disease Activity Marker in Inflammatory Bowel Disease Regulated by IL-17A, IL-22, and TNF-alpha and Modulated by IL23R Genotype Status. Inflamm Bowel Dis 2015; 21:2327-40.
18. Munakata K, Yamamoto M, Anjiki N, et al. Importance of the interferon-alpha system in murine large intestine indicated by microarray analysis of commensal bacteria-induced immunological changes. BMC Genomics 2008; 9:192.
19. Yamamoto M, Yamaguchi R, Munakata K, et al. A microarray analysis of gnotobiotic mice indicating that microbial exposure during the neonatal period plays an essential role in immune system development. BMC Genomics 2012; 13:335.
20. Price A E, Shamardani K, Lugo K A, et al. A Map of Toll-like Receptor Expression in the Intestinal Epithelium Reveals Distinct Spatial, Cell Type-Specific, and Temporal Patterns. Immunity 2018; 49:560-575 e6.
21. Kinnebrew M A, Buffie C G, Diehl G E, et al. Interleukin 23 production by intestinal CD103(+)CD11b(+) dendritic cells in response to bacterial flagellin enhances mucosal innate immune defense. Immunity 2012; 36:276-87.
22. Geremia A, Arancibia-Carcamo C V. Innate Lymphoid Cells in Intestinal Inflammation. Front Immunol 2017; 8:1296.
23. Li L L, Wang Y T, Zhu L M, et al. Inulin with different degrees of polymerization protects against diet-induced endotoxemia and inflammation in association with gut microbiota regulation in mice. Sci Rep 2020; 10:978.
24. Gurung M, Li Z, You H, et al. Role of gut microbiota in type 2 diabetes pathophysiology. EBioMedicine 2020; 51:102590.
25. Hanninen A, Toivonen R, Poysti S, et al. Akkermansia muciniphila induces gut microbiota remodelling and controls islet autoimmunity in NOD mice. Gut 2018; 67:1445-1453.
26. Cani P D, de Vos W M. Next-Generation Beneficial Microbes: The Case of Akkermansia muciniphila. Front Microbiol 2017; 8:1765.
27. Sorini C, Cosorich I, Lo Conte M, et al. Loss of gut barrier integrity triggers activation of islet-reactive T cells and autoimmune diabetes. Proc Natl Acad Sci USA 2019; 116:15140-15149.
28. Wang S, Ryan C A, Boyaval P, et al. Maternal Vertical Transmission Affecting Early-life Microbiota Development. Trends Microbiol 2020; 28:28-45.

1. A recombinant CRAMP-expressing food-grade bacterium which expresses a CRAMP peptide.

2. The recombinant CRAMP-expressing food-grade bacterium according to claim 1 wherein the bacterium is a recombinant CRAMP-expressing probiotic bacterium.

3. The recombinant CRAMP-expressing food-grade bacterium according to claim 2 wherein the recombinant CRAMP-expressing probiotic bacterium is selected in the group consisting of Bifidobacterium, Lactobacillus and Lactococcus.

4. The recombinant CRAMP-expressing food-grade bacterium according to claim 2 wherein the recombinant CRAMP-expressing probiotic bacterium is a L. lactis strain, a Lactobacillus casei strain, a L. lactis htrA strain, a Lactobacillus plantarum strain or a Bifidobacterium longum strain.

5. The recombinant CRAMP-expressing food-grade bacterium according to claim 2 wherein said recombinant CRAMP-expressing probiotic bacterium is a recombinant CRAMP-expressing Lactococcus Lactis deposited under the accession number CNCM I-5727.

6. The recombinant CRAMP-expressing food-grade bacterium according to claim 1, wherein the CRAMP peptide is a mouse CRAMP peptide and has a nucleic acid sequence as set forth in SEQ ID NO: 1 and an amino acids sequence as set forth in SEQ ID NO: 2, or is a human CRAMP peptide and has a nucleic acids sequence as set forth in SEQ ID NO: 3 and an amino acids sequence as set forth in SEQ ID NO: 4.

7. A method of treating an autoimmune disease in a subject in need thereof, comprising administering to the subject a therapeutically effective amount of the recombinant CRAMP-expressing food-grade bacterium of claim 1.

8. The method according to claim 7, wherein said recombinant CRAMP-expressing food-grade bacterium is a probiotic bacterium.

9. The method according to claim 7, wherein the autoimmune disease is an intra- and extra-intestinal dysbiosis-related disease.

10. The method according to claim 7 wherein the autoimmune disease is a type 1 diabetes, rheumatoid arthritis, multiple sclerosis or an autoimmune liver disease.

11. A therapeutic composition comprising a recombinant CRAMP-expressing food-grade bacterium.

12. A method of treating an autoimmune disease in a subject in need thereof comprising administering to the subject a therapeutically effective amount of the therapeutic composition of claim 11.

13. The method of claim 9, wherein the intra- and extra-intestinal dysbiosis-related disease is obesity.
